(19) United States
(12) Patent Application Publication
(10) Pub. No.: US 2014/0336135 A1
(43) Pub. Date: Nov. 13, 2014

(54) NOVEL USE OF RHODODENDRIN

(71) Applicant: Catholic University Industry Academy Cooperation Foundation, Seoul (KR)

(72) Inventors: Tae-Yoon Kim, Seoul (KR); Yoon Jea Jeon, Seongnam (KR)

(73) Assignee: Catholic University Industry Academy Cooperation Foundation, Seoul (KR)

(21) Appl. No.: 14/222,204

(22) Filed: Mar. 21, 2014

(30) Foreign Application Priority Data
May 13, 2013 (KR) 10-2013-0053616

(51) Int. Cl. A61K 31/7028

(52) U.S. Cl. CPC A61K 31/7028
USPC 514/25

(57) ABSTRACT

The present invention relates to a novel use of rhododendrin, and more particularly to a composition for antioxidant purposes or for the prevention or treatment of hyperproliferative skin disorders, which comprises rhododendrin as an active ingredient; and a method for inhibiting reactive oxygen species or for preventing and treating a hyperproliferative skin disorder, which comprises administering an effective amount of rhododendrin to a subject in need thereof. Rhododendrin is not cytotoxic and has an excellent effect of eliminating a large amount of reactive oxygen species (ROS) generated by TNF-α/IFN-γ or UV light, and thus is effectively used to prepare an antioxidant cosmetic composition or an antioxidant food composition. In addition, rhododendrin has the effect of inhibiting skin cell hyperproliferation and has an excellent effect of inhibiting a hyperproliferative skin disorder or skin hyperkeratinization mediated by Toll-like receptors (TLRs) in various skin diseases, particularly psoriasis.

### CROSS-REFERENCE TO RELATED APPLICATIONS

This application claims a priority to Korean Patent Application No. 10-2013-0053616, filed on May 13, 2013 with the Korean Intellectual Property Office, the entire disclosure of which is incorporated herein by reference.

### TECHNICAL FIELD

The present invention relates to a novel use of rhododendron. More particularly, the present invention relates to a composition for antioxidant purposes or for the prevention and treatment of hyperproliferative skin disorders, which comprises rhododendrin as an active ingredient, and a method for inhibiting reactive oxygen species or for preventing or treating hyperproliferative skin disorders, which comprises administering an effective amount of rhododendrin to a subject in need thereof.

### BACKGROUND ART

In a living body, reactive oxygen species (ROS) such as superoxide, hydroxyl radical and hydrogen peroxide are continuously produced, while antioxidant systems that remove ROS in response to ROS production are regulated. Thus, antioxidant defense systems are somewhat balanced with ROS production. However, as is known in the art, when the antioxidant defense systems are insufficient for inhibiting ROS production (that is, when oxidative stress occurs), DNA, proteins, lipids and other components, which constitute cells and tissues, can be damaged by ROS, and this oxidative stress is associated with the development of various kinds of cancers and age-related diseases. Thus, the research and development of functional foods containing antioxidant substances capable of preventing the development of diseases associated with oxidative stress is of increasing interest.

Rhododendrin is a secondary metabolite found in the family of Rhododendron. In the traditional Chinese medicine, Rhododendron has been well known to have analgesic and anti-inflammatory effects. Pharmacologically, it has been known to have diuretic, tonic and peptic effects, along with its effect against dysentery, vomiting and neuralgia. As a folk remedy, Rhododendron has been used for back pain caused by gastrointestinal convulsions, arthritis, menstrual irregularity, neuralgia and hypertension, along with being additionally used as a tonic, a diuretic and the like. However, there has been no report demonstrating the antioxidant activity of rhododendrin.

### DETAILED DESCRIPTION OF THE INVENTION

### Technical Problem

Accordingly, after having conducted studies to find novel antioxidant substances, the inventors of the present application have found that rhododendrin has excellent antioxidant activity, thereby completing the present invention.

Therefore, it is an object of the present invention to provide an antioxidant cosmetic composition comprising rhododendrin as an active ingredient.

Another object of the present invention is to provide an antioxidant food composition containing rhododendrin as an active ingredient.

Still another object of the present invention is to provide an antioxidant pharmaceutical composition containing rhododendrin as an active ingredient.

Still another object of the present invention is to provide a cosmetic composition for preventing and improving hyperproliferative skin disorders, which comprises rhododendrin as an active ingredient.

Still another object of the present invention is to provide a food composition for preventing and improving hyperproliferative skin disorders, which comprises rhododendrin as an active ingredient.

Still another object of the present invention is to provide a pharmaceutical composition for preventing and treating hyperproliferative skin disorders, which contains rhododendrin as an active ingredient.

Still another object of the present invention is to provide a method for inhibiting reactive oxygen species, which comprises administering an effective amount of rhododendrin to a subject in need thereof.

Still another object of the present invention is to provide a method for preventing and treating hyperproliferative skin disorders, which comprises administering an effective amount of rhododendrin to a subject in need thereof.

### Technical Solution

To achieve the above objects, the present invention provides an antioxidant cosmetic composition comprising rhododendrin as an active ingredient.

The present invention also provides an antioxidant food composition comprising rhododendrin as an active ingredient.

The present invention also provides an antioxidant pharmaceutical composition comprising rhododendrin as an active ingredient.

The present invention also provides a cosmetic composition for preventing and improving hyperproliferative skin disorders, which comprises rhododendrin as an active ingredient.

The present invention also provides a food composition for preventing and improving hyperproliferative skin disorders, which comprises rhododendrin as an active ingredient.

The present invention also provides a pharmaceutical composition for preventing and treating hyperproliferative skin disorders, which contains rhododendrin as an active ingredient.

The present invention also provides a method for inhibiting reactive oxygen species, which comprises administering an effective amount of rhododendrin to a subject in need thereof.

The present invention also provides a method for preventing and treating hyperproliferative skin disorders, which comprises administering an effective amount of rhododendrin to a subject in need thereof.

Hereinafter, the present invention will be described in further detail.

The present invention provides an antioxidant composition comprising rhododendrin as an active ingredient.

Rhododendrin according to the present invention can be represented by the following Formula 1:

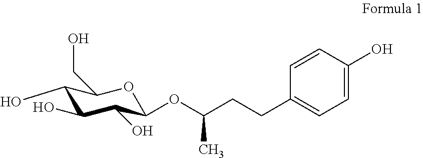

As used herein, the term “antioxidant” or “antioxidation” means preventing various oxidations from occurring in the body. Also, the term “antioxidant” or “antioxidation” is meant to include eliminating free radicals or reactive oxygen species (ROS), inhibiting the production of free radicals or reactive oxygen species (ROS), and indirectly promoting the elimination or inhibition of the production of free radicals or reactive oxygen species (ROS).

In a general state, reactive oxygen species (ROS) are produced in mitochondria during cell metabolism, while they physiologically function to protect cells from infectious agents, and play an important role as cell signaling factors. However, the accumulation of ROS causes oxidative stress and induces the dysfunction of cellular polymers such as proteins and DNAs.

As used herein, the term “reactive oxygen species” preferably means a superoxide radical (O2.-), hydrogen peroxide (H₂O₂), a hydroxyl radical (HO.), an oxygen single molecule (singlet oxygen, ¹O2), and mixtures thereof, but is not limited thereto. Most preferably, the term “reactive oxygen species” means hydrogen peroxide.

The present inventors isolated rhododendrin from an aqueous methanol fraction of the leaves of R. brachycarpum and examined whether the isolated rhododendrin influences the biological activity of HaCaT cells, and as a result, have found that rhododendrin has no cytotoxicity (see Example 2 and FIG. 1).

In addition, in the present invention, the ability of rhododendrin to eliminate intracellular ROS was measured using H2DCF-DA that is a hydrogen peroxide marker. As a result, it was found that rhododendrin effectively eliminated a large amount of ROS produced by TNF-α/IFN-γ or UV light, suggesting that rhododendrin has ROS scavenging activity (see Example 3).

Because rhododendrin is not cytotoxic and has excellent ROS scavenging activity as described above, rhododendrin can be used to prepare an antioxidant cosmetic composition, food composition and pharmaceutical composition.

The cosmetic composition of the present invention comprises rhododendrin as an effective component, and may be prepared in the form of basic cosmetics (lotions, cream, essence, cleansers such as cleansing foam and cleansing water, pack, body oil), coloring cosmetics (foundation, lip-stick, mascara, make-up base), hair care composition (shampoo, rinse, hair conditioner, hair gel) and soap with dermatologically acceptable excipients.

The above said excipients may include, but not limited thereto, skin softener, skin infiltration enhancer, colorant, odorant, emulsifier, thickener, or solvent. In addition, it is possible to add fragrance, a pigment, bactericidal agent, an antioxidant, a preservative, moisturizer and the like, while adding thickening agents, inorganic salts or synthetic polymers for improving physical properties. For example, in case of manufacturing a cleanser and a soap comprising the cosmetic composition according to the present invention, they may be prepared easily by adding rhododendrin to conventional cleanser and soap base. In case of manufacturing a cream, it may be prepared by adding rhododendrin to conventional oil-in-water cream base. Furthermore, it is possible to add a fragrance, a chelating agent, a pigment, an antioxidant, a preservative, and the like, and to add synthetic or natural proteins, minerals or vitamins for improving physical properties.

The content of rhododendrin in the cosmetic composition of the present invention is preferably 0.001-30 wt % based on the total weight of the composition, but is not limited thereto. If the content of rhododendrin in the cosmetic composition is less than 0.001 wt % based on the total weight of the composition, it cannot show a sufficient antioxidant effect, whereas if the content of rhododendrin is more than 30 wt %, it can cause problems in terms of the stability of the cosmetic composition, thus impairing the appearance of the cosmetic composition or diminishing the cosmetic effect of the cosmetic composition.

Meanwhile, the food composition of the present invention may include any kinds of forms such as functional food, nutritional supplement, health food, and food additives.

The food composition of the present invention may be prepared into various kinds of forms by the methods known in the art. For example, as a health food, rhododendrin may be prepared into tea, juice, and drink for drinking or may be prepared into liquefaction, granules, capsules, or powder for intake. Also, conventional active ingredients which are well known as having an antioxidant activity may be mixed with rhododendrin so as to prepare a composition. Further, for preparing a functional food, rhododendrin may be added to beverages (including alcoholic beverages), fruits, and their processed foods (e.g. canned fruit, bottled fruit, jam, marmalade etc.), fishes, meats, and their processed foods (e.g. ham, sausage, corn beef etc.), breads and noodles (e.g. Japanese noodle, buckwheat noodle, ramen, spaghetti, macaroni etc.), fruit juice, drinks, cookies, toffee, dairy products (e.g. butter, cheese etc.), vegetable oil, margarine, vegetable protein, retort food, frozen food, various seasonings (e.g. soybean paste, soybean sauce, sauce etc.). In addition, rhododendrin may be prepared in a form of powder or concentrated liquid for food additives.

Rhododendrin may be properly comprised by the form of a food composition, preferably in the range of 0.01 to 90% based on the total weight of the food composition. More preferably, the food composition of the present invention may be prepared particularly mixing conventional active ingredients which are well known as having an antioxidant activity with rhododendrin of the present invention.

The pharmaceutical composition of the present invention may comprise rhododendrin alone or together with one or more carrier, excipient, or diluent additionally. As used herein, the term “pharmaceutically acceptable” refers to a composition which is physiologically acceptable and does not cause any allergic reaction or similar effects thereto upon being administered to humans.

A pharmaceutically acceptable carrier, for example, carriers for parenteral or oral administration may be included. The carriers for the oral preparations may include lactose, starch, cellulose derivatives, magnesium stearate, stearic acid and the like.

In addition, the carriers for the parenteral preparations may include water, oil, saline, aqueous glucose and glycol, while additionally comprising stabilizers and preservatives. Examples of suitable stabilizers include antioxidants such as sodium hydrogen sulfite, sodium sulfite, and ascorbic acid. Examples of the preservatives include benzalkonium chloride, methyl- or prophyl-paraben, and chlorobutanol. The list of pharmaceutically acceptable carriers are disclosed in Remington's Pharmaceutical Sciences, 19th ed., Mack Publishing Company, Easton, Pa., 1995.

Preferably, the pharmaceutically acceptable carriers of the present invention may include glucose, saccharose, lactose, fructose, starch, dextrins, cyclodextrins, polyvinylpyrrolidone, alginic acid, tylose, silicic acid, cellulose, cellulose derivatives, mannitol, sorbitol, calcium carbonate, calcium phosphate, and the combination thereof.

The antioxidant pharmaceutical composition according to the present invention may be administered to mammals including humans via any routes, for instance orally or parenterally. The parenteral administration includes, but is not limited thereto, intravenous, intramuscular, intra-arterial, intraosseous, intrathecal, intracardiac, transdermal, subcutaneous, intraperitoneal, intranasal, gastrointestinal, topical, sublingual and rectal route. As described herein, the term “transdermal administration” means that the pharmaceutical composition of the present invention is administered to the cells or the skin of a subject, thereby the active ingredient contained in the antioxidant pharmaceutical composition of the present invention being delivered into the skin of a subject. For instance, the pharmaceutical composition of the present invention may be prepared into an injectable formulation, and then administered with a 30 gauge thin injection needle by lightly pricking the skin. Alternatively, it may be directly applied to the skin of a subject.

The pharmaceutical composition according to the present invention may be prepared into formulations for the above described oral or parenteral administration routes.

For oral formulation, the composition of the present invention may be formulated into powders, granules, tablets, pills, sugar-coated tablets, capsules, liquids, gels, syrups, slurries, suspensions and the like by using the methods known in the art. For instance, tablet or sugar-coated tablet type oral formulation may be prepared by mixing the active ingredient with solid excipients and being ground, followed by adding suitable additives to obtain a granule mixture. Examples of suitable excipients include sugars such as lactose, dextrose, sucrose, sorbitol, mannitol, xylitol, erythritol and maltitol; starches such as corn starch, wheat starch, rice starch and potato starch; celluloses such as cellulose, methyl cellulose, sodium carboxymethylcellulose and hydroxypropyl methylcellulose; and fillers such as gelatin and polyvinylpyrrolidone. If needed, the composition of the present invention may comprise cross-linked polyvinylpyrrolidone, agar, alginic acid or sodium alginate as a solutionizer. Further, the pharmaceutical composition according to the present invention may additionally comprise anti-cohesive agents, lubricants, wetting agents, flavoring agents, emulsifying agents and antiseptics.

In case of parenteral administration, the composition of the present invention may be formulated into injections, creams, lotions, ointments, oils, humectants, gels, aerosols and nasal inhaler by the methods known in the art. These formulations are described in the Remington's Pharmaceutical Science, 15th Edition, Chapter 87: Blaug, Seymour, 1975, Mack Publishing Company, Easton, Pa., which is well known in the pharmaceutical chemistry field.

The total effective amount of rhododendrin according to the present invention may be administered to a patient in a single dose or in multiple doses by a fractionated treatment protocol. The pharmaceutical composition of the present invention may contain variable amount of the active ingredient according to the severity of disease. The effective amount of rhododendrin is preferably about 0.01 μg to 1,000 mg/kg body weight/day, more preferably 0.1 μg to 100 mg/kg body weight/day. However, the dose of rhododendrin may be suitably determined by considering various factors, such as age, body weight, health condition, sex, disease severity, diet and excretion of a subject in need of treatment, as well as administration frequency and administration route. Considering the above factors, the skilled person in the art will be able to determine the appropriate effective amount of rhododendrin as an antioxidant agent. The pharmaceutical composition according to the present invention may not limit the type of formulations, administration routes, and administration methods as long as they show the desired effect according to the present invention.

Furthermore, the pharmaceutical composition according to the present invention may additionally comprise anti-cohesive agents, lubricants, wetting agents, flavoring agents, emulsifying agents and antiseptics.

For parenteral administration, the composition of the present invention may be formulated into injections by the methods known in the art. These formulations are described in the Remington's Pharmaceutical Science, 15th Edition, Chapter 87: Blaug, Seymour, 1975, Mack Publishing Company, Easton, Pa., which is well known in the pharmaceutical chemistry field.

Also, the pharmaceutical composition of the present invention may be administered together with conventional compounds which are known to possess an antioxidant activity.

The present invention also provides a cosmetic composition and a food composition for preventing and improving a hyperproliferative skin disorder, which comprises rhododendrin as an active ingredient, and a pharmaceutical composition for preventing and treating a hyperproliferative skin disorder, which comprises rhododendrin as an active ingredient.

Herein, rhododendrin, a cosmetic composition, a food composition and a pharmaceutical composition are as described above.

“Proliferating disorders of skin” means a condition in which the skin cells proliferate due to various stimuli or inflammations, leading to the thickening of the skin's keratinous layer. As described in the present invention, “proliferating disorders of skin” preferably means a hyperproliferative skin disorder. One exemplary symptom of the hyperproliferative skin disorder is hyperkeratosis in which the skin's keratinous layer becomes abnormally thick so that the surface of the skin becomes hard and thick.

The hyperproliferative skin disorder according to the present invention include any skin disease that involves skin hyperkeratinization. Preferably, the hyperproliferative skin disorder may be a hyperkeratosis disease mediated by Toll-like receptors. More preferably, the hyperproliferative skin disorder may be selected from the group consisting of psoriasis, atopic dermatitis, acne, eczema, seborrheic dermatitis, lichen planus, pityriasis rosea, acute varioliform eruption, pityriasis lichenoides chronica, parapsoriasis, and lichen striatus. Most preferably, the hyperproliferative skin disorder may be psoriasis.

Rhododendrin has the effect of inhibiting skin cell proliferation. Particularly, rhododendrin has an excellent effect of inhibiting skin cell proliferation mediated by Toll-like receptors (TLRs). The specific interaction between rhododendrin and Toll-like receptor 7 (TLR7) is first disclosed in the specification of the present application. This is described in detail in the Examples of the present application.

In an example according to the present invention, in order to examine the effect of rhododendrin on the inhibition of skin cell proliferation, mice with psoriasis induced by imiquimod were treated with rhododendrin, and the skin tissues of the mice were then observed. As a result, it was shown that the expression of TLR7 that increased by imiquimod, the thickness of the epidermal and dermal layers, and skin hyperkeratinization, significantly decreased.

In another example according to the present invention, in order to examine the effect of rhododendrin on the TLR7 signaling pathway, keratinocytes were treated with imiquimod and rhododendrin, and the activity of the TLR7 marker NF-kB in the keratinocytes was measured. As a result, it was found that the down signaling of TLR7 was activated by imiquimod, whereas the activity of TLR7 in the group treated with rhododendrin was inhibited. In addition, it was found that the activity of caveolin, a marker against psoriasis, was protected by rhododendrin.

The present invention also provides a method for inhibiting reactive oxygen species, which comprises administering an effective amount of rhododendrin to a subject in need thereof.

Further, the present invention provides a method for preventing and treating a hyperproliferative skin disorder, which comprises administering an effective amount of rhododendrin to a subject in need thereof.

As used herein, the term “subject” means animals, preferably mammals, particularly animals including humans. In addition, the subject may also be a cell, a tissue or an organ derived from an animal. The subject may be a patient in need of treatment. As used herein, the expression “subject in need thereof” means a subject who is in need of the inhibition of reactive oxygen species or has a hyperproliferative skin disorder.

As used herein, the term “effective amount” means the amount of rhododendrin that exhibits an effective effect, that is, the effect of inhibiting reactive oxygen species or the effect of preventing and treating a hyperproliferative skin disorder, in a subject in need thereof.

The inventive method for inhibiting reactive oxygen species or for preventing and treating a hyperproliferative skin disorder, which comprises administering an effective amount of rhododendrin to a subject in need thereof, may preferably be a method that uses an antioxidant composition or a composition for preventing and treating a hyperproliferative skin disorder, which comprises rhododendrin as an active ingredient. Herein, the composition is as described above.

Thus, rhododendrin according to the present invention may be administered in itself or in the form of various compositions and formations containing the same as described above. Preferably, rhododendrin may be administered until the desired effect, that is, the effect of inhibiting reactive oxygen species or the effect of preventing and treating a hyperproliferative skin disorder, will be obtained. Rhododendrin according to the present invention may be administered by various routes known in the art. Specifically, rhododendrin may be administered orally or parenterally, for example, buccally, intramuscularly, intravenously, transdermally, intra-arterially, intramedullarily, intradurally, intraperitoneally, intranasally, intravaginally, intrarectally, sublingually or subcutaneously, or may be administered into the gastrointestinal tract, a mucosa or a respiratory organ. For example, rhododendrin according to the present invention may be administered by applying a composition containing it directly to the skin, injecting an injectable formulation comprising rhododenrin subcutaneously into the skin by a 30-gauge syringe needle, or lightly pricking the skin with a syringe needle containing it. Preferably, a composition comprising rhododendrin may be applied directly to the skin.

In addition, the composition of the present invention may preferably be formulated as any parenteral administration form selected from the group consisting of an injectable formulation, a cream formation, a lotion formulation, an ointment formulation, an oil formulation, a moisturizing formulation, a gel formulation and an aerosol formulation.

### Advantageous Effects

Rhododendrin according to the present invention is not cytotoxic and has an excellent effect of eliminating a large amount of ROS generated by TNF-α/IFN-γ or UV light. Thus, it can be effectively used to prepare an antioxidant cosmetic composition, an antioxidant food composition or an antioxidant pharmaceutical composition.

In addition, rhododendrin has the effect of inhibiting skin cell proliferation. Particularly, it has an excellent effect of inhibiting skin hyperkeratinization mediated by Toll like receptors (TLRs) in various skin diseases including psoriasis.

### BRIEF DESCRIPTION OF THE DRAWINGS

FIG. 1 shows the results of measuring cell viability at various concentrations of rhododendrin (Y-axis: values relative to 100% for a control group not treated with rhododendrin; X-axis: concentration of rhododendrin (μM)).

FIG. 2 shows the results of observing the ROS scavenging activity of rhododendrin by a confocal microscope (PBS: PBS-treated control group; TNF-α/IFN-γ: test group stimulated with TNF-α and IFN-γ; DMSO: control group not treated with rhododendrin; RDN: test group treated with rhododendrin).

FIG. 3 shows the results of measuring the ROS scavenging activity of rhododendrin by fluorescence (DMSO: PBS-treated control group; DMSO-(TNF-α+IFN-γ): control group stimulated with TNF-α and IFN-γ; ROR 20 μM-(TNF-α+IFN-γ): test group treated with 20 μM of rhododendrin, and then stimulated with TNF-α and IFN-γ).

FIG. 4 shows the results of observing the ROS scavenging activity of rhododendrin by a confocal microscope (PBS: control group treated with PBS; UVB-DMSO: control group treated with UV light; UVB-Rhodo: test group treated with rhododendrin, and then stimulated with UV light).

FIG. 5 shows the results of measuring the ROS scavenging activity of rhododendrin by fluorescence (-: control group treated with PBS; +: control group treated with UV light; ROR 20 μM: test group treated with rhododendrin, and then stimulated with UV light).

FIG. 6 shows the results of visually observing the skin condition of psoriasis-induced mouse models treated in various manners (Ror: rhododendrin).

FIG. 7 shows the results of histologically examining the skin tissue excised from psoriasis-induced mouse models treated in various manners (Ror: rhododendrin).

FIG. 8 shows the thicknesses of the epidermal layer (left) and dermal layer (right) of the skin tissue of psoriasis-induced mouse models treated in various manners (Ror: rhododendrin).

FIG. 9 shows the results of examining the TRL7 mRNA level in the skin tissue of psoriasis-induced mouse models treated in various manners.

FIG. 10 shows the results of examining the IL-17 mRNA level in the skin tissue of psoriasis-induced mouse models treated in various manners.

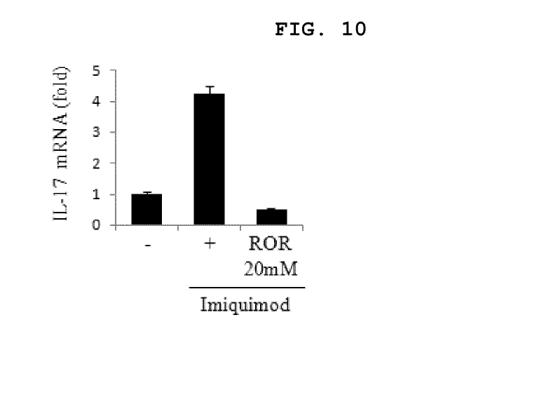

FIG. 11 shows the results of examining the CCL17 mRNA level in the skin tissue of psoriasis-induced mouse models treated in various manners.

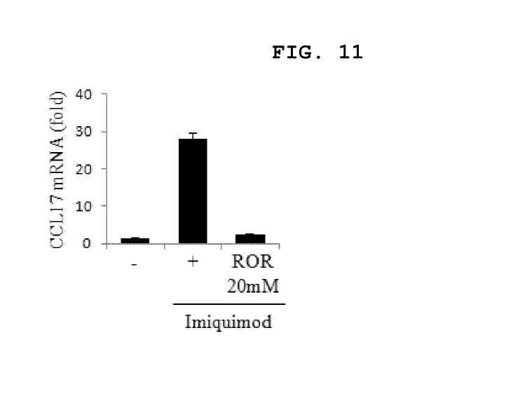

FIG. 12 shows the results of examining the effects of rhododendrin on the inhibition of TLR7 expression and TLR7 down signaling in keratinocytes by Western blot analysis for NF-kB p65 that is the TLR7 marker.

FIG. 13 shows the results of examining the effects of rhododendrin on the inhibition of TLR7 expression in keratinocytes by fluorescence staining for NF-kB p65 that is the TLR7 marker (ImQ: group treated with imiquimod; ImQ+20 μM: group treated with imiquimod and 20 μM rhododendrin).

FIG. 14 shows the results of examining the caveolin-protecting effect of rhododendrin in keratinocytes by fluorescence staining (IMQ: group treated with imiquimod; IMQ+Ror20: group treated with imiquimod and 20 μM rhododendrin).

### MODE FOR INVENTION

Hereinafter, the present invention will be described in detail with reference to examples. It is to be understood, however, that these examples are for illustrative purposes only and are not intended to limit the scope of the present invention.

### Example 1

### Isolation and Purification of Rhododendron

Powdered R. brachycarpum leaves were repeatedly extracted with dichloromethane (10 L×3) and methanol (10 L×3). The combined crude extract (52.8 g) was partitioned between water (H₂O) and n-butanol (24.1 g), and the latter fraction was re-partitioned between n-hexane (4.9 g) and 15% aqueous methanol (MeOH; 20.9 g). An aliquot of 15% aqueous MeOH layer (6.0 g) was subjected to reversed-phase vacuum flash chromatography using sequential mixtures of H₂O and MeOH as eluents (elution order: 50%, 40%, 30%, 20%, 10% aqueous MeOH, and 100% MeOH; 100% acetone, and 100% ethyl acetate). The fraction (1,080 mg) eluted with 40% aqueous MeOH from flash chromatography was separated by semi-preparative high-performance liquid chromatography (HPLC; YMC ODS-A column, 5 m, 250×10 mm, 50% aqueous MeOH) to yield rhododendrin. Final purification was then performed by reversed-phase HPLC (YMC ODS-A column, 5 m, 250×4.6 mm, 60% aqueous MeOH) to produce 141.2 mg of rhododendrin.

### Example 2

### Examination of Cytotoxicity of Rhododendrin

HaCaT cells were purchased from CLS Cell Line Service GmbH (Eppelheim, Germany). HaCaT cells were seeded at a density of 2×10⁴ cells/well in 96-well plates. After a 12 hours incubation, the cells were incubated with various concentrations of rhododendrin for 24 hours. Then, cells were incubated with 3-(4,5-dimethylthiaol-2-yl)-2,5-diphenyltetrazolium bromide (MTT) reagent for 2-4 hours at 37° C. To calculate cell viability, MTT signals were quantified with a microplate reader (BioTek, Winooski, Vt., USA) at wavelength of 540 nm.

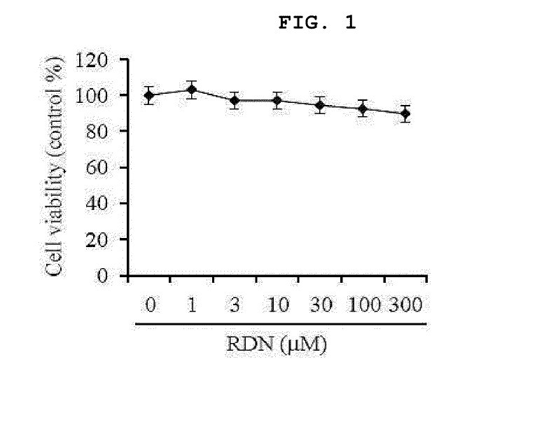

The results are shown in FIG. 1. From the results in FIG. 1, it can be seen that rhododendrin has no cytotoxicity.

### Example 3

### ROS Scavenging Activity (Antioxidant Effect of Rhododendrin

3-1: Induction of ROS Production by TNF-α/IFN-γ

HaCaT cells were seeded at a density of 3×10⁵ cells/well in 6-well plates for 12 hours. Cells were washed with serum-free media, treated with 20 μm rhododendrin for 1 hour, and then stimulated with 10 ng/ml of TNF-α and 100 U/ml of IFN-7 for 3 hours. The cells were stabilized in Hanks' balanced salt solution (HBSS) for 30 minutes, stained with 10 μM 2′,7′-dichlorofluorescin diacetate (H2DCF-DA) at 37° C. for 30 minutes, and ROS signal was then analyzed with confocal microscopy with emission at 513 nm and excitation at 488 nm. Also, the fluorescence of the cells was measured with a microplate reader (Synergy, BIO TEK, US).

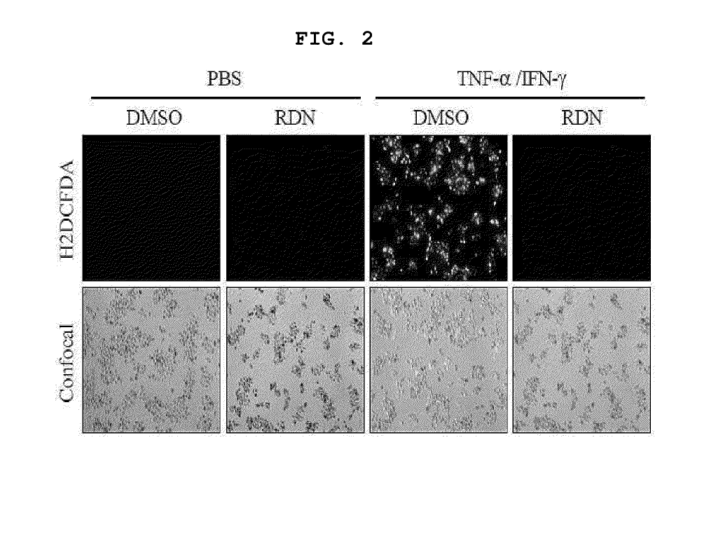

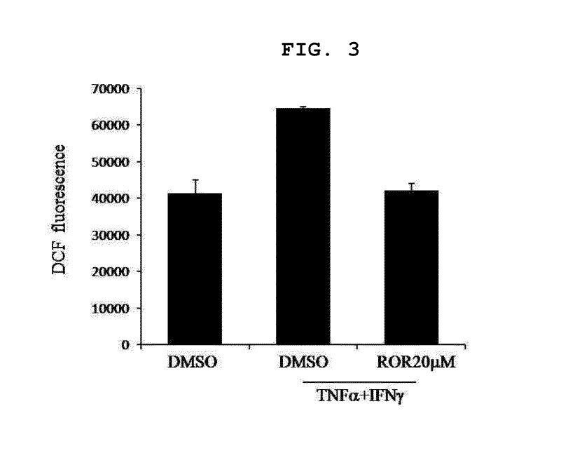

The results of the measurement are shown in FIGS. 2 and 3. As can be seen therein, in the group stimulated with TNF-α/IFN-γ but not treated with rhododendrin, a large amount of ROS were generated, whereas in the group treated with rhododendrin and the group treated with PBS (phosphate buffered saline), no fluorescence was detected (see FIG. 2). In addition, as can be seen in FIG. 3, the production of DCF-fluorescence in the group treated with rhododendrin decreased to that in the control group not treated with TNF-α/IFN-γ. This suggests that rhododendrin has an excellent ability to scavenge ROS.

3-2: Induction of ROS by UV Light—Effect on Inhibition of Intracellular ROS Induced by UV Irradiation

Human keratinocyte HaCaT cells (obtained from professor N. Fusenig, Cancer Research, Germany) were placed in a 60-mm culture dish at a density of 5×10⁵ cells (1×10⁶ cells for a 100 mm culture dish), and then cultured in 10% FBS (fetal bovine serum)-containing DMEM (Dulbecco's modified Eagle's medium) for 24 hours.

After culture, the cells were treated with 20 μM rhododendrin for 1 hour, and then washed three times with PBS (phosphate buffered saline). To induce ROS (reactive oxygen species), the cells were irradiated with 100 J/m² of UV light from a UV lamp (FSX 24 T12/UVB/HO, Pansol™ USA). Immediately after UV irradiation, the cells were washed with PBS and cultured for 4 hours in 1% FBS-containing DMEM medium supplemented with homoisoflavanone. Then, the cells were treated with 25 μM DCFH-DA (2,7-dichlorofliuorescin diacetate) in HBSS (Hank's balanced salt solution, BioWhittaker, CAMBREX, USA) buffer for 30 minutes, and then washed three times with PBS. Next, the measurement of ROS production in the cells by H2DCF-DA staining was performed in the same manner as described in Example 3-1.

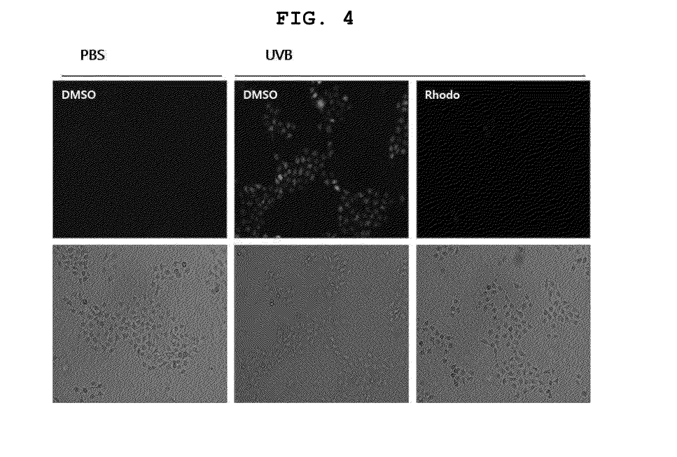

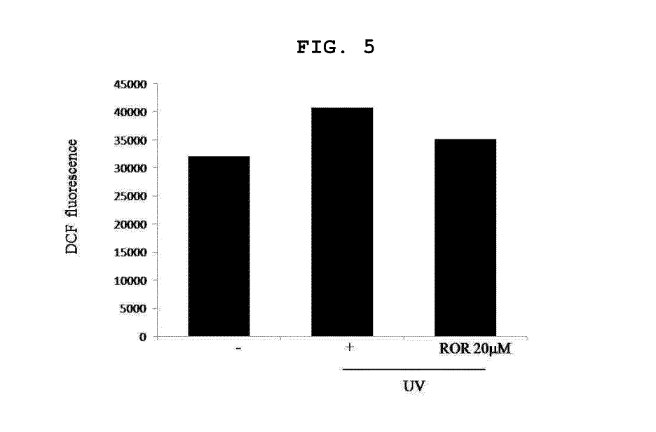

The results of the measurement are shown in FIGS. 4 and 5. As can be seen therein, a large amount of fluorescence was found in the control group stimulated with UV light, whereas only very minute fluorescence spots were found in the group treated with rhododendrin (see FIG. 4). Also, the amount of DCF-fluorescence as measured was significantly lower in the group treated with rhododendrin than in the group not treated with rhododendrin (see FIG. 5).

### Example 3-3

### Statistical Analysis

Experimental data are expressed as the mean±standard error of means (SEM). Differences between the control and variable conditions were determined by a two-tailed Student's t-tests and were considered to be significant at p<0.05.

### Example 4

### Effect of Rhododendrin on Inhibition of Skin Hyperkeratinization

Psoriasis, which is a typical skin hyperkeratinization disease, was induced in mice in order to examine the effect of rhododendrin on the inhibition of a skin hyperkeratinization disease. When imiquimod, which is used as an antiviral therapeutic agent, is applied to the skin, it can cause skin psoriasis, but the expression of Toll-like receptor 7 (TLR7) that mediates the action of imiquimod in the skin has not been sufficiently known. This experiment was focused on inducing psoriasis in mice by imiquimod and examining the specific interaction between TLR7 and rhododendrin.

4-1: Examination of the Effect on the Inhibition of Hyperkeratinization in Psoriasis-Induced Model

Tissues were excised from mice having psoriasis induced by imiquimod, and then the effect of rhododendrin on the inhibition of hyperkeratinization in the tissues was examined by histological analysis and qRT (quantitative real-time)-PCR analysis.

Specifically, in order to induce psoriasis by imiquimod, the hairs on the back of 8-week-old mice (C57BL/6, Central Animal Laboratory Inc.) were removed by using hair removal cream. The back of the mice was pre-treated daily with 20 mM rhododendrin before 1 hour, and then imiquimod (3.125 mg) was applied to the back at an one-day interval for 7 days. On the last day of application, the tissues were collected.

Histological examination of the mice skin was performed in the following manner. The mouse skin tissues were fixed in PBS containing 4% paraformaldehyde for 24 hours, washed with tap water, dehydrated with ethanol, and then embedded in paraffin to make a paraffin block. The paraffin block was cut with a microtome into 4 μm-thick sections, which were then placed on a glass slide and treated with xylene to remove wax (paraffin). Then, the tissue sections were dehydrated with ethanol and stained with hematoxylin-eosin (H&E). Histological analysis was performed using a fluorescence attached microscope (Olympus, Japan).

RNA isolation and qRT (quantitative real-time)-PCR analysis were performed in the following manner. Total RNAs were isolated from the tissues using an RNeasy Mini Kit (Qiagen, Valencia, Calif., USA) according to the manufacturer's instructions, and then synthesized into cDNA using QuantiTect Reverse Transcription Kit (Qiagen) according to the manufacturer's instructions. qRT-PCR was performed using Rotorgene™6000 (Corbett life Science) with KAPA™SYBR FAST q-PCR Master Mix(2×) universal. The relative amount of mRNAs was determined by AACt method using GAPDH as an internal control. PCR was performed under the following conditions: 95° C. for 5 min; 35 cycles, each consisting of 96° C. for 20 sec, 60° C. for 20 sec and 72° C. for 20 sec; and one cycle at 72° C. for 5 min. Also, the primer set used in the qRT-PCR is shown in Table 1 below.

TABLE 1

| Gene | Catalog number | Manufactured by |
|---|---|---|
| IL-17 | QT00103278 | Qiagen |
| CCL17 | QT00131572 | Qiagen |
| TLR7 | MQP037300 | GeneCopoeia |

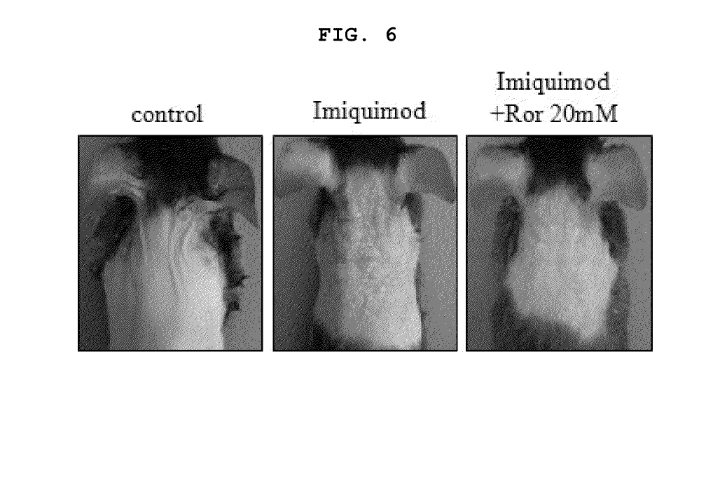

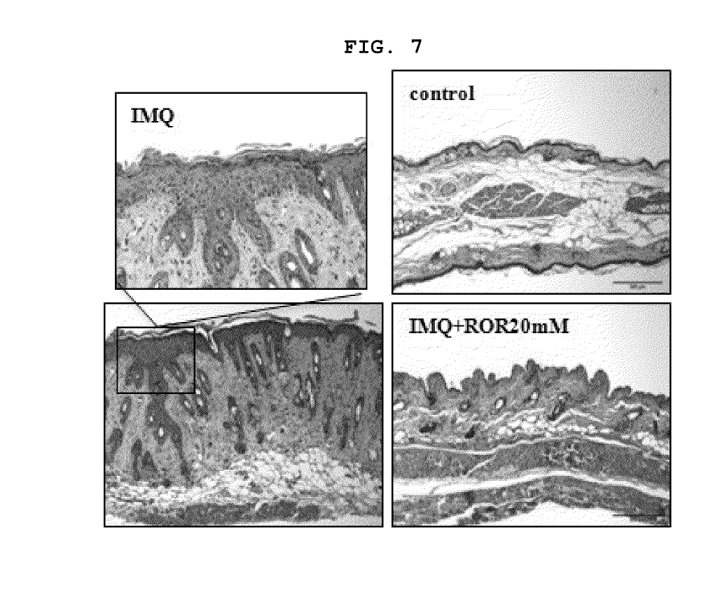

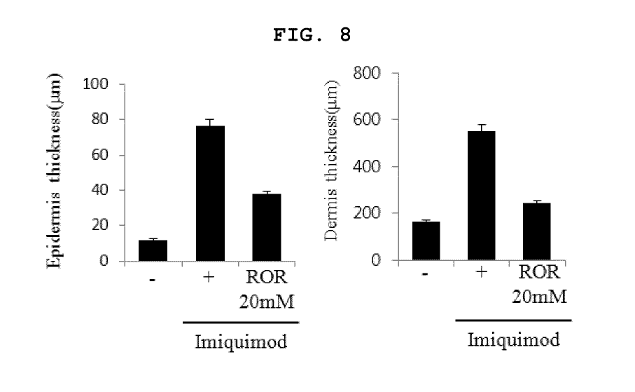

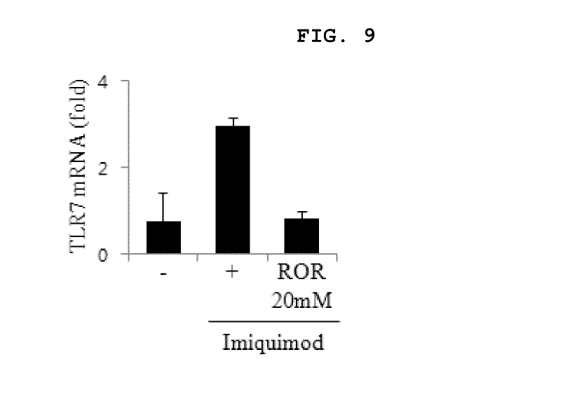

As a result, as can be seen in FIGS. 6 to 8, the thickness of the epidermal and dermal layers in the group treated with imiquimod became thicker, and when this group was treated with rhododendrin, the induction of psoriasis significantly decreased (visual observation of the back tissue; a decrease in the thickness of the epidermal and dermal layers). In addition, it was found that the expression of TLR, which is the maker of imiquimod, was significantly lower in the rhododendrin-treated group than in the control group (see FIG. 9).

4-2: Examination of the Effect of Rhododendrin on TLR7 Signaling Pathway

Keratinocytes were treated with imiquimod, and then TLR7 signaling in the cells was examined by Western blot analysis and immune fluorescence staining. The detailed experimental methods are as follows.

Primary keratinocytes collected from the foreskin of a healthy subject were cultured. Human keratinocytes were cultured in keratinocyte serum-free medium (K-SFM) supplemented with epidermal growth factor (EGF) and bovine pituitary extract (Gibco BRL, Rockville, Md.). When the cells reached to 70% confluency, the cells were washed twice with PBS and cultured in K-SFM for 24 hours. To induce the differentiation of the keratinocytes, 1.2 mM calcium was added to the cells which were then cultured for 1, 3, 7 and 14 days, respectively.

Western blot analysis was performed in the following manner. Serum-starved keratinocytes were treated with various concentrations of rhododendrin for 1 hour, and then stimulated by treatment with imiquimod for different time periods. The cell pellets were lysed in ice-cold RIPA buffer (Roche Applied Science, Indianapolis, Ind., USA) containing protease inhibitor cocktail at 4° C. for 30 minutes. Protein samples were isolated by SDS-PAGE (sodium dodecylsulfate-polyacrylamide gel electrophoresis) and analyzed by immunoblotting. Briefly, the protein on the SDS polyacrylamide gel was transferred to a nitrocellulose membrane (Amersham, UK). Then, the transferred membrane was blocked with 5% skim milk-containing buffer and treated with Tris-buffered saline (TBS, pH 7.4) containing 0.1% Tween 20 (TBST) at room temperature for 1 hour. Then, the membrane was treated with primary antibody (phospho-NF-kB p65 (Ser536), IRAK1, IRAK4) and allowed to stand still at 4° C. overnight. Next, the membrane was probed with horseradish peroxidase-conjugated secondary antibody (horseradish peroxidase (HRP)-conjugated goat anti-rabbit and anti-mouse antibodies, Life Technologies (Grand Island, N.Y., USA)), and then incubated with enhanced chemiluminescence detection kit (iNtRON Biotechnology, Korea). A signal was detected using X-ray film development or Image-Analyzer System (LAS-3000, Fujifilm, Japan) in a dark room.

In addition, the expression patterns of TLR7, NF-kB (a marker of TLR7) and caveolin (a marker of psoriasis) were analyzed by immunofluorescence in the following manner.

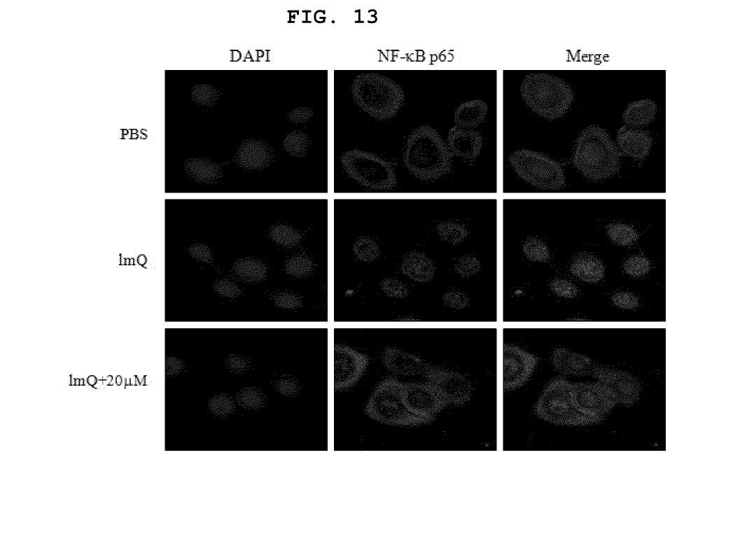

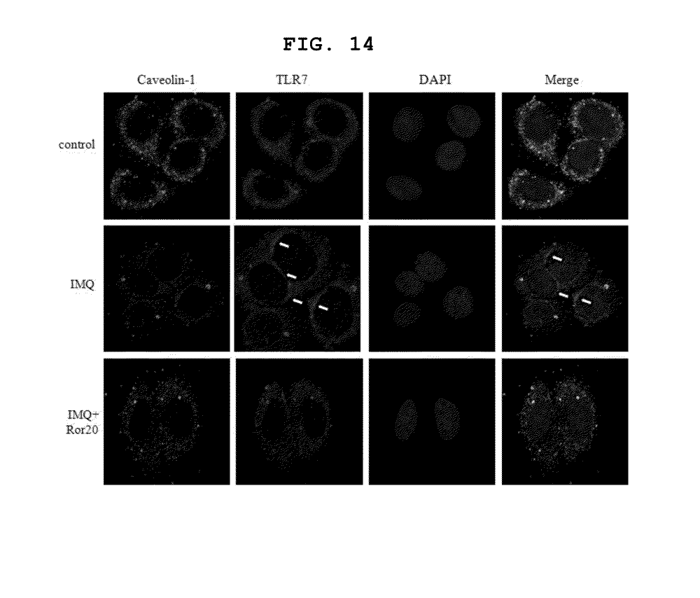

Primary keratinocytes (5×10⁵ cells/well) were pre-treated with rhododendrin for 1 hour, and then stimulated with imiquimod for 1 hour. The signal of immunostained NF-kB p65 was analyzed by the method described in Y. Kim. et. al., Free Radical Biology and Medicine; 51(11): 1985-1995, 2011. Briefly described, the cells were fixed in 4% paraformaldehyde, permeabilized with 0.5% Triton X-100, and then blocked with PBS (containing 1% bovine serum albumin). For immunostaining, the cells were incubated with anti-NF-kB p65 antibody for 2 hours, and then washed three times with 1% PBS and incubated with Alexa Fluor 568 anti-rabbit IgG antibody (Molecular Probe, Eugene, Oreg., USA) for 1 hour. The immunostained NF-kB p65 was observed with a confocal fluorescence microscope (Carl Zeiss, Thornwood, N.Y., USA), and the nucleus was counter-stained with Hoechst (Molecular Probe, Eugene, Oreg., USA) (FIG. 13). Also, in the same manner as above, the cells were incubated with anti-TLR7 antibody and anti-caveolin-1 antibody for 2 hours, and then washed three times with a blocking buffer and incubated with Alexa Fluor 568 anti-rabbit IgG antibody (Molecular Probe, Eugene, Oreg., USA) and Alexa Fluor 488 anti-mouse IgG antibody (Molecular Probe, Eugene, Oreg., USA) for 1 hours (FIG. 14).

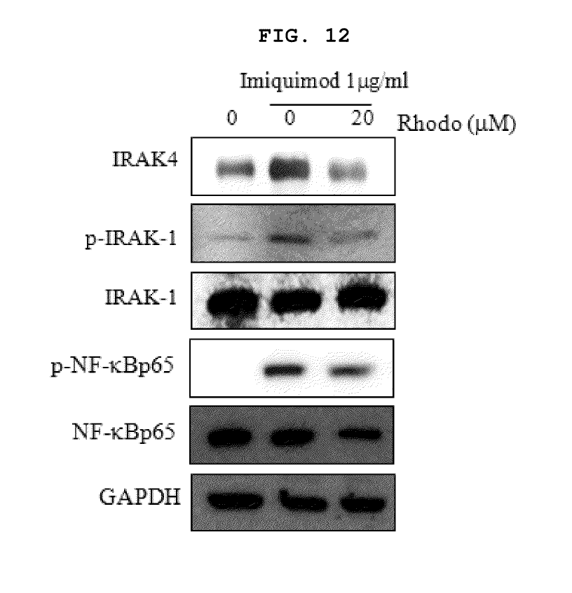

It was found that, when the keratinocytes were treated with imiquimod, the down signaling of TLR7 in the cells was activated, whereas the activity of TLR7 in the group treated with rhododendrin was inhibited, and the activity of NF-kB (maker of TLR7) in the rhododendrin-treated group was also inhibited (see FIGS. 12 and 13). Such results suggest that rhododendrin inhibits the expression of TLR7, thus preventing the down signaling of TLR7.

Also, as shown in FIG. 14, TLR7 was located in the endosome, and when the cells were fluorescence-stained with caveolin-1 (a marker of endosome), colors appeared at the same location. When the cells were treated with imiquimod, the expression of TLR7 in the cells increased and the expression of caveolin decreased. Such results are consistent with a report indicating that the expression of caveolin (a marker of psoriasis) decreases in psoriasis patients (Campbell L, Laidler P, Watson R, Kirby B, Griffiths C E, Gumbleton M, Down-regulation and altered spatial pattern of caveolin-1 in chronic plaque psoriasis, Br J Dermatol. 2002 October; 147(4): 701-9.).

### INDUSTRIAL APPLICABILITY

As described above, rhododendrin is not cytotoxic and has an excellent effect of eliminating a large amount of ROS generated by TNF-α/IFN-γ or UV light. Thus, it can be effectively used to prepare an antioxidant cosmetic composition, an antioxidant food composition or an antioxidant pharmaceutical composition, and thus is highly industrially applicable. In addition, rhododendrin has the effect of inhibiting skin cell hyperproliferation and has an excellent effect of inhibiting skin hyperkeratinization that is mediated by Toll like receptors (TLRs) in various skin diseases, particularly psoriasis, and thus can be highly effectively used for the preparation of an antioxidant cosmetic composition, an antioxidant food composition and an antioxidant pharmaceutical composition.

1. A method for inhibiting reactive oxygen species, the method comprising administering to a subject in need thereof an effective amount of rhododendrin represented by the following formula 1:

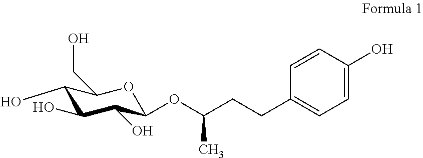

2. The method of claim 1, wherein the reactive oxygen species is selected from the group consisting of a superoxide radical, hydrogen peroxide, a hydroxy radical, singlet oxygen, and mixtures thereof.

3. The method of claim 1, wherein rhododendrin is administered using a parenteral formulation selected from the group consisting of an injectable formulation, a cream formulation, a lotion formulation, an ointment formulation, an oil formulation, a moisturizing formulation, a gel formulation and an aerosol formulation.

4. A method for preventing and treating a hyperproliferative skin disorder, the method comprising administering to a subject in need thereof an effective amount of rhododendrin represented by the following formula 1:

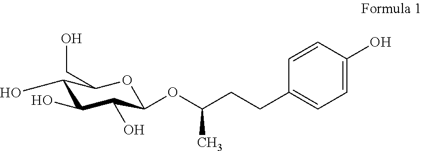

5. The method of claim 4, wherein the hyperproliferative skin disorder is selected from the group consisting of psoriasis, atopic dermatitis, acne, eczema, seborrheic dermatitis, lichen planus, pityriasis rosea, acute varioliform eruption, pityriasis lichenoides chronica, parapsoriasis, and lichen striatus.

6. The method of claim 4, wherein rhododendrin is administered using a parenteral formulation selected from the group consisting of an injectable formulation, a cream formulation, a lotion formulation, an ointment formulation, an oil formulation, a moisturizing formulation, a gel formulation and an aerosol formulation.
